(19) United States
(12) Patent Application Publication
(10) Pub. No.: US 2014/0188198 A1
(43) Pub. Date: Jul. 3, 2014

(54) HYPODERMIC NEEDLE WITH FIBEROPTIC EXTENSION

(71) Applicant: Donald SPECTOR, New York, NY (US)

(72) Inventor: Donald SPECTOR, New York, NY (US)

(21) Appl. No.: 13/954,063

(22) Filed: Jul. 30, 2013

Related U.S. Application Data
(60) Provisional application No. 61/747,337, filed on Dec. 30, 2012.

(51) Int. Cl. A61N 5/06

(52) U.S. Cl. CPC A61N 5/0601
USPC 607/92

(57) ABSTRACT

A device for treating disease has a hypodermic needle having a hollow shaft and an insertion tip, at least one fiberoptic filament extending through the needle to the insertion tip, and a UV light source connected to the fiberoptic filament. The UV light source is adapted to transmit UV light at a wavelength of 280-100 nm. The hypodermic needle is inserted into a patient until the insertion tip is located in an area to be treated, and the UV light is supplied to the at least one fiberoptic filament, so that the light travels along the filament to the area to be treated.

### CROSS-REFERENCE TO RELATED APPLICATION

This application claims priority under 35 U.S.C. 119(e) of U.S. Provisional Application Ser. No. 61/747,337, filed on Dec. 30, 2012.

### BACKGROUND OF THE INVENTION

1. Field of the Invention

The present invention is relates to a fiberoptic cable disposed inside a hypodermic needle for applying UV light to a diseased area of the body in order to treat the disease.

2. The Prior Art

Medical procedures for treating diseased tissue often involve cutting into the patient and removing the tissue. These procedures can have many complications and can involve a painful and prolonged recovery. It would be desirable to be able to treat the diseased tissue without causing unnecessary physical trauma to the patient.

UV (ultraviolet) light is often used to kill bacteria and is also a known cell killer. IR (infrared) light is also used to kill diseased cells, such as cancerous cells.

### SUMMARY OF THE INVENTION

It is an object of the invention to provide a device and method for treating disease. by using UV light supplied to a diseased area without causing unnecessary trauma to the patient. It is another object of the invention to provide a device and method for treating disease that is portable and simple and economic to use.

These and other objects are accomplished by a device comprising a hypodermic needle having a hollow shaft and an insertion tip, at least one fiberoptic filament extending through the needle to the insertion tip, and a UV light source connected to the fiberoptic filament. The UV light source is adapted to transmit UV light at a wavelength of 280-100 nm, which is the range in which the UV light interferes with a cell's DNA and can destroy diseased cells and bacteria. Studies have shown that UV light has intense peaks at UV wavelengths of 253.7 nanometers (nm) and a lesser peak at 184.9 nm. Research has shown that the optimum UV wavelength range to destroy bacteria is between 250 nm and 270 nm.

In one embodiment, the least one fiberoptic component is made up of a plurality of fiberoptic filaments forming a fiberoptic cable.

In a preferred embodiment, the UV light source is a light emitting diode (LED). The LED is small and lightweight, so makes an ideal light source for use with the invention, which is intended to be a portable device. Other sources of UV light could also be used.

The present invention also comprises a method for treating disease using a device comprising a hypodermic needle having a hollow shaft and an insertion tip, at least one fiberoptic filament extending through the needle to the insertion tip, and a UV light source connected to the fiberoptic filament, the UV light source adapted to transmit UV light at a wavelength of 280-100 nm. The involves inserting the hypodermic needle into a patient until the insertion tip is located in an area to be treated; and supplying UV light to the at least one fiberoptic filament, so that the light travels along the filament to the area to be treated.

The UV light can be supplied in either a continuous beam or in a pulsed sequence. If a pulsed sequence is used, the sequence can include at least three pulses.

The method can be repeated as many times as necessary to achieve sufficient treatment of the patient.

### BRIEF DESCRIPTION OF THE DRAWINGS

Other objects and features of the present invention will become apparent from the following detailed description considered in connection with the accompanying drawings. It is to be understood, however, that the drawings are designed as an illustration only and not as a definition of the limits of the invention.

In the drawings, wherein similar reference characters denote similar elements throughout the several views:

FIG. 1 shows a cross-sectional view of a device according to the invention; and

FIG. 2 shows the device in use on a patient.

### DETAILED DESCRIPTION OF THE PREFERRED EMBODIMENT

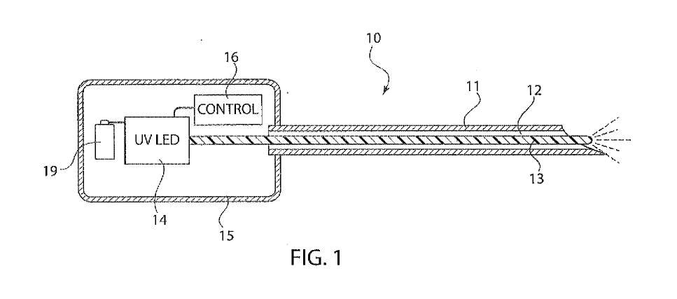

Referring now in detail to the drawings and, in particular, FIG. 1 shows the device 10, which comprises a hypodermic needle 11 with a hollow interior 12, through which a fiberoptic cable 13 extends. Needle 11 could be made in various sizes, depending on the size of cable 13. Cable 13 is made of a plurality of fiber optic filaments encased in a covering. Cable 13 is connected to a UV light source formed by a Light Emitting Diode (LED) 14, which is encased in a housing 15 connected to needle 11. LED 14 creates UV light, which travels down cable 13 to its end. LED 14 creates UV light in a wavelength of 250-270 nm, which is the wavelength at which the DNA inside bacteria and cells becomes disrupted, thus causing cell death. LED 14 can be configured to emit a constant beam of light, or can be a pulsed beam. A controller 16 can be connected to LED 14 to control the type of beam being emitted, as well as to turn the beam on and off.

Power to the LED 14 could be supplied by a battery 19i incorporated into housing 15, or by any conventional power supply means. Use of a battery means that device 10 is portable and can be used anywhere, without the need for an electrical connection.

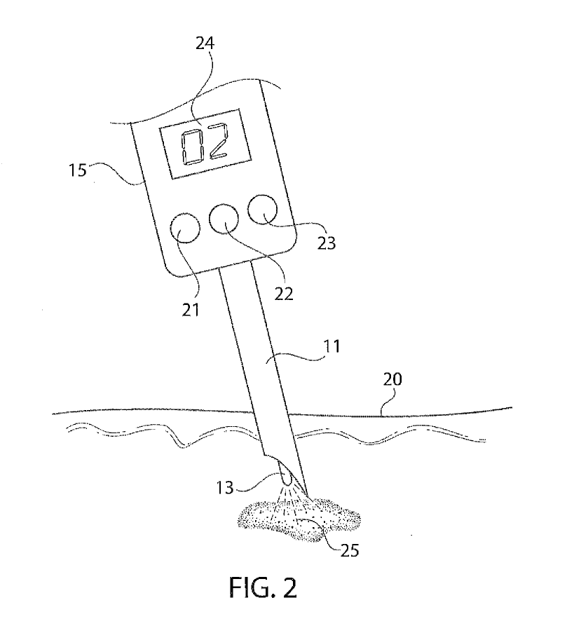

The device 10 in use is shown in FIG. 2. In use, needle 11 is inserted into a patient 20 to a depth where needle 10 is in the vicinity of the area to be treated. The area to be treated could have a bacterial infection, cancer or other disease. After insertion, power is supplied via controller 16 to LED 14, to send UV light 25 through cable 13. Housing 15 is supplied with controllers 21 for turning the light on, 22 for turning the power off, and 23 creating a pulsed light emissions. A display screen 24 can show the duration of the light supplied, either in time or in number of pulses, as well as any other desired information.

Once the treatment is completed, the needle 11 is removed from the patient 20. The treatment can be repeated as many times as necessary and in multiple locations to achieve elimination of the disease.

Accordingly, while only a few embodiments of the present invention have been shown and described, it is obvious that many changes and modifications may be made thereunto without departing from the spirit and scope of the invention.

What is claimed is:

1. A device for transmitting UV light to a target, comprising:
a hypodermic needle having a hollow shaft and an insertion tip;
at least one fiberoptic filament extending through the needle to the insertion tip; and
a UV light source connected to the fiberoptic filament, the UV light source adapted to transmit UV light at a wavelength of 280-100 nm.

2. The device according to claim 1, wherein the light source is adapted to transmit UV light at a wavelength of 250-270 nm.

3. The device according to claim 1, wherein said at least one fiberoptic filament comprises a plurality of fiberoptic filaments forming a fiberoptic cable.

4. The device according to claim 1, wherein the light source is a light emitting diode (LED).

5. The device according to claim 1, further comprising a controller connected to the UV light source for controlling power to the light source.

6. The device according to claim 1, wherein the light source is powered by a battery.

7. A method for treating disease using a device comprising a hypodermic needle having a hollow shaft and an insertion tip, at least one fiberoptic filament extending through the needle to the insertion tip, and a UV light source connected to the fiberoptic filament, the UV light source adapted to transmit UV light at a wavelength of 280-100 nm, the method comprising the following steps:
inserting the hypodermic needle into a patient until the insertion tip is located in an area to be treated; and
supplying UV light to the at least one fiberoptic filament, so that the light travels along the filament to the area to be treated.

8. The method according to claim 7, wherein the step of supplying includes supplying the UV light in a pulsed sequence.

9. The method according to claim 8, wherein the sequence includes at least three pulses.

10. The method according to claim 7, further including repeating the steps of inserting and suppling.
